(19) United States
(12) Patent Application Publication
(10) Pub. No.: US 2007/0055229 A1
(43) Pub. Date: Mar. 8, 2007

(54) In tunnel electrode for sealing intracardiac defects

(76) Inventors: Stephanie M. Kladakis, Watertown, MA (US); James J. Scutti, Arlington, MA (US); Carol A. Devellian, Topsfield, MA (US); Helen S. Liu, Bedford, MA (US)

Correspondence Address: KIRKPATRICK & LOCKHART NICHOLSON GRAHAM LLP, One Lincoln Street, BOSTON, MA, 02111-2950, US

(21) Appl. No.: 11/516,315

(22) Filed: Sep. 6, 2006

Related U.S. Application Data
(60) Provisional application No. 60/734,558, filed on Nov. 8, 2005.
(60) Provisional application No. 60/714,374, filed on Sep. 6, 2005.

(51) Int. Cl. A61B 18/18

(52) U.S. Cl. 606/41

(57) ABSTRACT

The present invention provides methods and devices for sealing intracardiac defects, such as a patent foramen ovale (PFO) utilizing an electrode positioned in the lumen of the defect such as the tunnel of a PFO.

### RELATED APPLICATIONS

This application claims the benefit of and priority to U.S. provisional application 60/714,374, filed Sep. 6, 2005, and U.S. provisional application 60/734,558, filed Nov. 8, 2005, the disclosures each of which are incorporated by reference herein.

### FIELD OF THE INVENTION

The invention relates to a method and apparatus for closing intracardiac defects via a percutaneous transvascular route. More specifically, the invention relates to an apparatus that delivers an energy-delivering electrode into the tunnel of a patent foramen ovale to substantially close the tunnel, and to a method for substantially closing the tunnel of a patent foramen ovale by withdrawing an energized RF electrode from the tunnel.

### BACKGROUND OF THE INVENTION

The human heart is divided into four compartments or chambers. The left and right atria are located in the upper portion of the heart and the left and right ventricles are located in the lower portion of the heart. The left and right atria are separated from each other by a muscular wall, the interatrial septum, and the ventricles are separated by the interventricular septum.

Either congenitally or by acquisition, abnormal openings (holes or shunts) can occur between the chambers of the heart or between the great vessels, causing inappropriate blood flow. Such deformities are usually congenital and originate during fetal life when the heart forms from a folded tube into a four chambered, two-unit, i.e., atrial and ventricular, system. The septal deformities result from the incomplete formation of the septum, or muscular wall, between the left and right chambers of the heart and can cause significant problems.

One such septal deformity or defect, a patent foramen ovale (PFO), is a persistent tunnel with a flap-like opening in the wall between the right atrium and the left atrium of the heart. Since left atrial pressure is normally higher than right atrial pressure, the flap typically stays closed. Under certain conditions, however, right atrial pressure exceeds left atrial pressure, creating the possibility for right to left shunting of venous blood that can allow blood clots and other toxins to enter the systemic circulation. This is particularly problematic for patients who have deep vein thrombosis or clotting abnormalities.

Devices for sealing an intracardiac defect such as a PFO in a patient are well known in the art. Prior art devices typically provide a catheter with an electrode that is applied to the external tissue of the PFO on the right atrial side. The electrode is energized and the tissues forming the tunnel on the right atrial side of the atrial septum are generally damage in a non-specific pattern, i.e., more tissue than just the tissue lining the tunnel of the PFO is damaged. In other words, pinpoint application of energy to cardiac tissues within the tunnel is not possible with these prior art devices. In addition, without a means for stabilizing the catheter in a beating heart during these procedures, prior art devices are likely to extend the scope of cardiac tissue damage beyond the tissues of the tunnel. The present invention described below addresses these drawbacks.

### SUMMARY OF THE INVENTION

The invention in one aspect relates to an apparatus for substantially closing the tunnel of a PFO. In one embodiment, the apparatus includes a catheter having a proximal end, a distal end and a lumen and an elongated member including an electrode. In a further embodiment, the apparatus includes a vacuum cone that stabilizes the apparatus to the patient's cardiac tissues while the electrode is energized for delivery energy to the cardiac tissues.

In a particular embodiment of the invention, the elongated member includes one or more projections such as one or more filaments projecting from the distal end portion or distal tip of the elongated member. The one or more filaments include a fixed end and a free end. The filaments may include one or more electrodes, e.g., an RF electrode located anywhere along the filament including, for example, at the free end of the filament. The one or more filaments may be, for example, curvilinear or straight. Additionally, the one or more filaments may be flexible, or, alternatively, rigid. In a particular embodiment, the fixed end of each of the filaments is equidistant from the distal tip of the elongated member. Alternatively, the fixed ends of each of the filaments are dispersed along the length of the elongated member. The distal end portion of the elongated member comprises 10-40% of the length of the elongated member, in particular, 15%, 20%, or 30% of the length of the elongated member. In yet another embodiment, the fixed end of the one or more filaments is positioned at the distal tip of the elongated member.

According to the invention, the electrodes may be positioned anywhere along the length of the filament from the fixed end to the free end and/or anywhere along the length of the elongated member. The electrodes may deliver radio frequency energy, cryogenic energy, laser energy, ultrasonic energy, resistive heat energy, or microwave energy, for example.

In another aspect, the invention relates to a method for closing the tunnel of a PFO. In one embodiment, the method includes the step of providing an apparatus including a catheter having a lumen extending from a proximal end to a distal end, and an elongated member comprising an electrode, the elongated member being slideably movable in the lumen of the catheter. The elongated member is deployed from the end of the catheter into the tunnel and the one or more electrodes are energized. The elongated member and electrode are withdrawn in a proximal direction from the tunnel of the PFO while the electrode is energized thereby applying energy to the cardiac tissues in the tunnel of the PFO from the distal end of the tunnel to the proximal end of the tunnel to seal the tunnel while the elongated member is withdrawn. In yet another embodiment of the method of the invention, the electrode is energized intermittently as an energized-de-energized cycle while the electrode and the elongated member are withdrawn from the tunnel of the PFO. In one embodiment of the method of the invention, a vacuum cone is placed over the cardiac tissues and a vacuum is applied to stabilize the apparatus on the cardiac tissue while energy is applied to substantially seal the PFO.

As used throughout, to “substantially seal” or “substantially close” the PFO it is meant that a stable tissue bridge will be formed across the PFO, which will withstand physiological pressures. A substantially closed or sealed PFO, however, may still have one or more small gaps or openings which will in at least some cases close over time via the healing process.

### BRIEF DESCRIPTION OF THE DRAWINGS

While the present invention is capable of embodiment in various forms, there is shown in the drawings and will be hereinafter described, an exemplification of the invention, and is not intended to limit the invention to the specific embodiments disclosed.

In the drawings like reference characters generally refer to the same parts throughout the different views. Also, the drawings are not necessarily to scale, emphasis, instead generally being placed upon illustrating the principles of the invention.

FIG. 1 is a perspective cutaway view of a heart illustrating a PFO.

FIG. 2 illustrates a plan view of the apparatus for closing intracardiac defects according to an illustrative embodiment of the invention.

FIG. 3 illustrates a portion of the elongated member of the apparatus illustrated in FIG. 2 according to an illustrative embodiment of the invention.

FIG. 4 illustrates a portion of the elongated member of the apparatus illustrated in FIG. 2 according to another illustrative embodiment of the invention.

FIG. 5 illustrates a portion of the catheter and the elongated member of the apparatus illustrated in FIG. 2 according to an illustrative embodiment of the invention.

FIG. 6 illustrates a portion of the catheter and the elongated member including filaments of the apparatus illustrated in FIG. 2 according to an illustrative embodiment of the invention.

FIG. 7 illustrates a portion of the catheter and the elongated member including filaments of the apparatus illustrated in FIG. 2 according to another illustrative embodiment of the invention.

FIG. 8 illustrates a portion of the elongated member including an abrasive surface according to an illustrative embodiment of the invention.

FIG. 9 illustrates a portion of the elongated member including two shafts and an abrasive surface according to an illustrative embodiment of the invention.

FIGS. 10A-10D illustrate a method for closing a PFO according to an illustrative embodiment of the invention.

### DESCRIPTION OF THE INVENTION

The embodiments of the present apparatus described below have in common a movable elongated member having an electrode along its distal end portion. The apparatus is introduced into the patient needing treatment via the percutaneous, transvascular route into the right atrium of the patient's heart. The advantages of the present invention include a slideably movable electrode for delivery of energy within the tunnel of the patient's PFO. The apparatus and method described herein has the further advantage of being minimally invasive and atraumatic compared to conventional procedures requiring a thoracotomy.

The present invention features systems, apparatus, and related methods, described below, for closing cardiac openings, such as, for example, a PFO. Throughout the description, the terms proximal and distal refer to the position of elements relative to the operator of the exemplary apparatus. Proximal is that portion of the delivery system or apparatus closer to the operator and distal is that portion of the delivery system or apparatus further away from the operator.

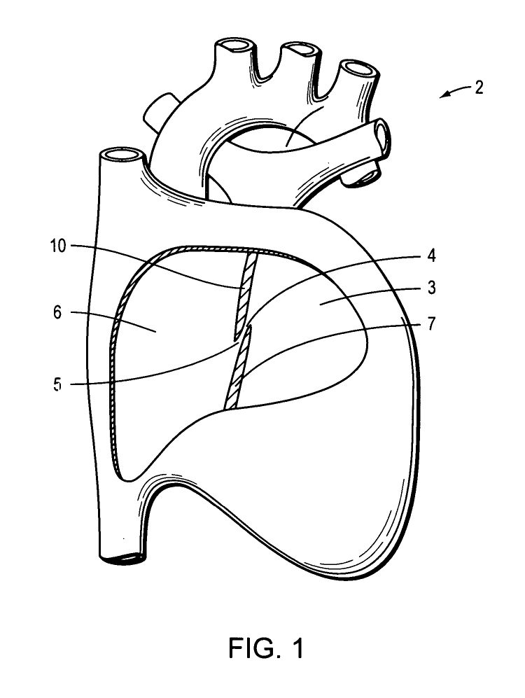

FIG. 1 depicts a cutaway view of a heart 2. The heart 2 includes a septum 4 that divides a right atrium 6 from a left atrium 3. The septum 4 includes a septum secundum 10 and a septum primum 7. An exemplary cardiac opening, a patent foramen ovale 5, that is to be corrected by the system and related method of the present invention is located between the septum secundum 10 and the septum primum 7. The PFO 5 provides an undesirable fluid communication between the right atrium 6 and the left atrium 3 and, under certain conditions, allows for the shunting of blood and toxins carried by the blood between the right atrium 6 and the left atrium 3. The PFO 5 typically has a tunnel. If the PFO 5 is not closed or obstructed in some manner, a patient is placed at higher risk for an embolic stroke in addition to other circulatory abnormalities.

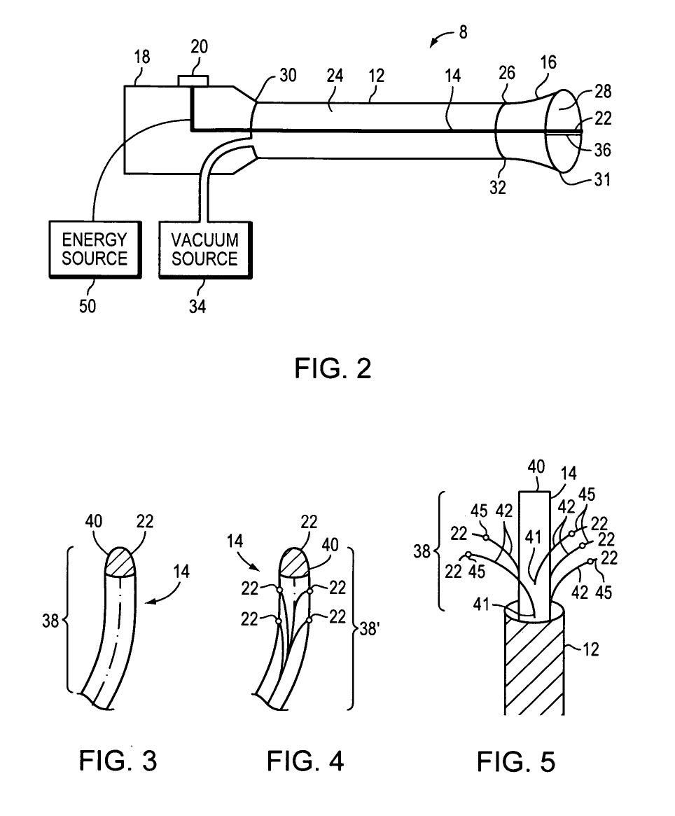

In one aspect, the invention is directed to an apparatus for closing a PFO. One example of the present invention will now be explained with reference to FIG. 2. FIG. 2 shows an exemplary delivery system 8 which includes a handle 18 with an actuator 20, a catheter 12 with a axially disposed lumen 24, an elongated member 14 slideably disposed inside the lumen 24, and at least one energy delivery element, for example, electrode 22 disposed on the elongated member 14.

In another embodiment, the delivery system 8 further includes a vacuum cone 16 that is used to apply negative pressure to stabilize the catheter 12 while delivering the elongated member 14 into the PFO tunnel. The vacuum applied to stabilize the catheter 12 may also have the advantage of collapsing the tunnel of the PFO.

With continued reference to FIG. 2, in a particular embodiment, the vacuum cone 16 is disposed at the distal end 26 of the catheter 12. The exemplary catheter 12 extends from a proximal end 31 at the handle 18 to a distal end 26. The vacuum cone 16 includes a lumen 28 in communication with the lumen 24 of the catheter 12.

A cone, as used herein, means any tubular shape or any tubular shape including a flared end. In a preferred embodiment, the cone 16 includes a tube having a flared end, i.e., the diameter of the distal end 30 of the cone 16 is greater than the diameter of the proximal end 32 of the cone 16. The flare may begin at the proximal end 32 of the cone 16 and extend gradually to the distal end 30 of the cone 16 as illustrated in FIG. 2, or, alternatively, the flare may begin anywhere along the long axis of the cone 16 and extend to the distal end 30 of the cone 16 (not shown). The cross-section of the distal end 30 of the cone 16 may be circular, oval, U-shaped or any other shape suitable for interfacing with intracardiac tissue. According to the invention, the vacuum cone 16 and a source of negative pressure may or may not be present. In one embodiment of the invention, the apparatus does not include a vacuum or a source of negative pressure.

With continued reference to FIG. 2, in one embodiment, the cone 16 includes a single lumen 28 in fluid communication with the lumen 24 of the catheter 12. Alternatively, the cone 16 has a plurality of lumens 28 (not shown). One of the plurality of lumens 28 houses the elongated member 14. At least one other of the plurality of lumens 28 is in fluid communication with the lumen 24 of the catheter 12.

Referring still to FIG. 2, in a preferred embodiment, a vacuum source 34 is operatively joined to the lumen 24 of the catheter and the lumen 28 of the cone 16.

With further reference to FIG. 2, the elongated member 14 extends through the lumen 24 of catheter 12. In one embodiment, the distal end 36 of the elongated member 14 transitions from a first position, where the distal end 36 of the elongated member 14 is housed within the lumen 24 of the catheter 12 to a second position, where the distal end 36 of the elongated member 14 is positioned outside of the lumen 24 of the catheter 12 and beyond the distal end 26 of the catheter 12, or in embodiments including a cone 16, beyond the distal end of the cone 16.

According to one embodiment of the invention, the elongated member 14 is operatively joined to the actuator 20 on the handle 18. In an alternative embodiment, the catheter 12 is operatively joined to the actuator 20 on the handle 18. In one embodiment the elongated member 14 transitions from the first position to the second position by extending the elongated member 14 operatively joined to the elongated member 14, distally while the catheter 12 is stationary. For example, the elongated member 14 may be operatively joined to the actuator 20 on the handle 18. Alternatively, the elongated member 14 transitions from the first position to the second position as the catheter 12, operatively joined to the actuator 20, is withdrawn proximally while the elongated member 14 is stationary.

Referring now to FIG. 3, the electrode 22 may be disposed anywhere along a distal end portion 38 of the elongated member 14. The distal end portion 38 includes about 1-30%, preferably 10-20%, more preferably 15% of the length of the elongated member 14 at its distal end. In one embodiment, for example, the electrode 22 is disposed on the distal tip 40 of the distal end portion 38. Alternatively, a plurality of electrodes 22 may be disposed along the surface of the distal end portion 38 of the elongated member 14. Referring to FIG. 4, in yet another embodiment, an electrode 22 is positioned on the distal tip 40, and one or more electrodes 22 are positioned along the surface of the elongated member 12 at its distal end portion 38.

The electrodes 22 are operatively connected to an energy source 50. The energy generated by the energy source 50 includes but is not limited to radio frequency energy, cryogenic energy, laser energy, ultrasonic energy, resistive heat energy, microwave energy and the like.

Referring now to FIG. 5, in one embodiment, the elongated member 14 includes at least one projection 42, e.g., a filament 42. The filament 42 has a fixed end 41 joined to the distal end portion 38 of the elongated member 14. A free end 45 is on the opposite end of the filament 42 from the fixed end 41. One or more electrode 22 may be disposed at the free end 45 of the filament 42 or anywhere along the surface from the free end 45 to the fixed end 41 of the filament 42.

The elongated member 14 may include any combination of filaments 42 and any number of electrodes 22 on the distal end portion 38 or on the distal tip 40 of the elongated member 14 and/or on the free end 45 of the one or more filaments 42 or anywhere along the length of one or more filaments 42.

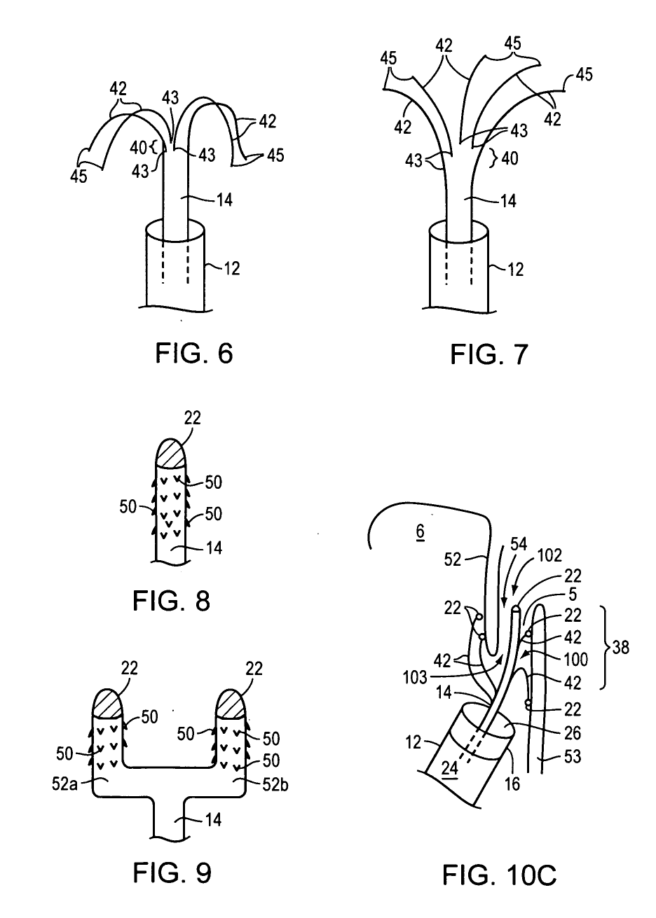

Referring to FIG. 6, in yet another embodiment according to the invention, one or more filaments 42 extend from the distal tip 40 of the elongated member 14. In a particular embodiment, the free end 45 of filament 42 reverses direction whereby the free end 45 of the filament 42 is directed proximally towards the proximal handle 18. In an alternative embodiment, the free end 45 of the filament 42 may be distal to the fixed end 41 or proximal to the fixed end 41.

Referring to FIG. 7 in another embodiment according to the invention, one or more filaments 42 extend from the distal tip 40 of the elongated member 14. In a particular embodiment, for example, one or more filaments 42 fan out from the distal tip 40 of the elongated member 14. For example, the free end 45 of the one or more filaments 42 is distal to the fixed end 41 and the distal tip 40 of the elongated member 14.

With respect to FIGS. 6 and 7, one or more electrodes 22 may be disposed in any number and in any combination anywhere along the filament 42 from the free end 45 to the fixed end 41 or at the free end 456 of the filament 42. Any combination of positions and numbers of filaments and electrodes is contemplated by the invention and the invention is not limited to the embodiments illustrated.

Referring now to FIG. 8, in one embodiment the elongated member 14 includes one or more spikes, teeth, or other types of abrasive materials 50 disposed on the surface of the distal end portion 38 of the elongated member 14. Typically the abrasive material 50 is disposed on the distal end portion 38 of the elongated member 14 proximal to at least one electrode 22. Alternatively, the abrasive material 50 is located proximal to all electrodes 22. The cross-sectional shape of the elongated member 14 is oval shape or, alternatively, circular, for example. Other shapes may also be used depending on the shape of the defect, e.g., a PFO, into which the elongated member 14 will be inserted.

In an alternative embodiment, referring now to FIG. 9, the elongated member 14 branches into more than one shaft 52, for example two shafts 52a, and 52b each shaft 52a, 52b including at least one electrode 22, and at least one abrasive material 50. For example, the elongated member 14 may be y-shaped as shown in FIG. 9, trident shaped (not shown), or have four or more shafts 52 (not shown). The abrasive material 50 is located proximal to at least one electrode 22 or to all electrodes 22.

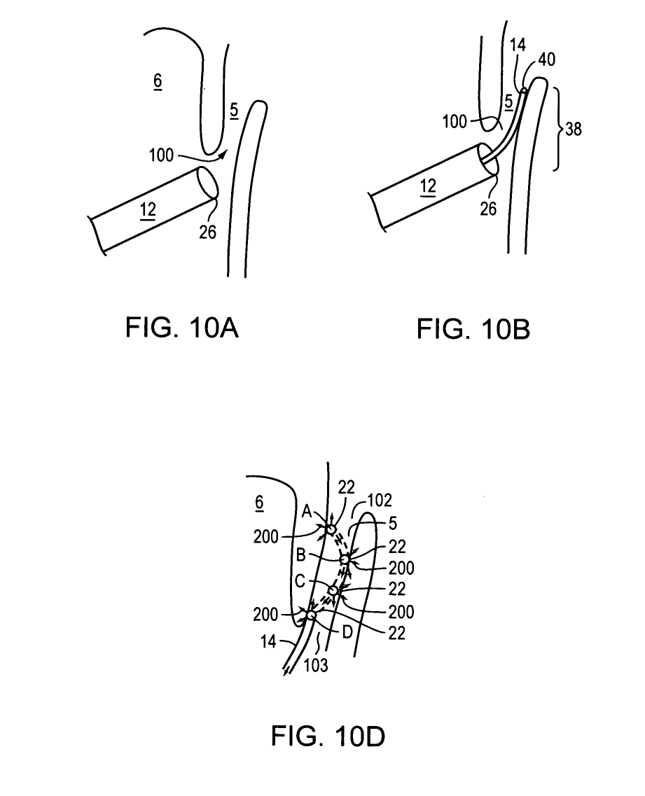

In another aspect, the invention is directed to a method for treating the tunnel of a PFO in the cardiac tissues of a patient. FIGS. 10A-10D demonstrate a method for treating the tunnel of a PFO according to one embodiment of the method of the invention. For example, Referring to FIG. 10A, the apparatus 8 according to the invention described above is introduced into a patient via a percutaneous, transvascular route, such as, e.g., via the femoral vein (not shown). The distal end 26 of the catheter 12 is introduced into the right atrium 6 and placed near or touching the tissues surrounding the entrance 100 to the tunnel of the PFO 5. In one embodiment, illustrated in FIG. 10B, while the catheter 12 touches the cardiac tissue near the entrance 100 of the PFO 5 and is kept stationary, the elongated member 14 transitions from a first position, (not shown), within the catheter 12 to a second position where at least the distal end 40 of the elongated member 14 is extended beyond the distal end 26 of the catheter 12 and deployed into the tunnel of the PFO 5. In a particular embodiment, the distal end portion 38 of the elongated member 14 is deployed into the tunnel of the PFO 5. In another embodiment, the catheter 12 is extended distally into the tunnel of the PFO 5 while holding the elongated member 14 in a first position. The elongated member 14 is then transitioned from a first position to a second position and therefore deployed inside the tunnel of the PFO 5 by withdrawing the catheter 12 proximally. In another embodiment according to the invention, while the distal end 26 of the catheter 12 or, e.g., the vacuum cone 16 described above with respect to FIG. 2 touches the cardiac tissue at the entrance 100 of the PFO 5, negative pressure from a vacuum source is applied from the vacuum cone 16 to the tissues surrounding the entrance 100 to the PFO 5. The catheter 12 is stabilized while the distal end 40 of the elongated member 14 is transitioned from a first position within the catheter 12 to a second position, i.e., beyond the distal end 31 of the cone 16 and deployed into the tunnel of the PFO 5.

In one embodiment illustrated in FIG. 10C, one or more electrodes 22 are positioned on cardiac tissues within the PFO tunnel 5 and one or more electrodes 22 are positioned on cardiac tissues outside the tunnel of the PFO 5, e.g., at the entrance 100 of the PFO. Alternatively, all of the electrodes 22 are positioned within the tunnel of the PFO 5.

After the electrodes 22 are positioned appropriately, energy is supplied to each electrode 22 simultaneously, sequentially, or in any order as determined by the operator to induce sufficient tissue damage to substantially close the tunnel of the PFO 5. Closure may occur immediately or over several days, weeks or months. The applied energy may be, for example, radio frequency, microwave, ultrasound, resistive, laser, heat or cryogenic, in an amount sufficient to alter the tissues in the tunnel of the PFO 5 so that the tissues substantially seal together to close the PFO 5.

In one embodiment according to the invention, after the elongated member 14 is placed in a distal position within the tunnel of the PFO 5, the elongated member 14 is withdrawn proximally, i.e., in a direction toward the right atrium, from position A within the tunnel of the PFO 5, closest to the left atrial side of the tunnel, to position B, to position C, to position D, closer to the right atrial side of the tunnel, and so on, while energy 200 is directed intermittently or continuously from one or more electrodes 22 to the tissues within the tunnel of the PFO 5 thereby causing tissue damage progressing from the distal end 102 of the tunnel towards the proximal end 103 of the tunnel. For example, after the electrode 22 is placed in a distal position in the tunnel of the PFO 5, the electrode cycles through an energized state followed by the electrode 22 being de-energized. The electrode 22 is then withdrawn proximally but not removed from the tunnel until the energized-de-energized cycle is repeated for example, at least once. Alternatively, the electrode 22 is continuously energized as the electrode 22 is withdrawn proximally from the tunnel of the PFO 5. In yet another embodiment, at least one electrode 22 on the elongate member 14 cycles at least once through the energized-de-energized cycle as the electrode 22 is withdrawn proximally from the tunnel of the PFO 5 and at least one other electrode 22 on the elongated member 14 is continuously energized as the electrode 22 is withdrawn from the tunnel of the PFO. The energized-de-energized cycles may occur at different times for one or more electrodes 22, or the energized-de-energized cycle may occur simultaneously for all of the electrodes 22. The number of positions to which the one or more electrodes 22 are moved in the tunnel of the PFO 5 is not limited to that illustrated.

Alternatively, according to the method of the invention, the elongated member 14 illustrated in FIG. 8 is withdrawn from the inside of the PFO tunnel while the abrasive materials 50 on the surface of the elongated member 14 abrade the tissues in the PFO tunnel. Energy is directed continuously to the PFO tissue from the electrodes 22 distal to the abraded tissue thereby inducing tissue adhesion that progresses from the distal end 102 towards the proximal end 103 of the tunnel of the PFO 5.

Alternatively, the elongated member 14 illustrated in FIG. 9 is deployed inside the PFO tunnel by withdrawing catheter 12 proximally while the elongated member 14 is stationary while positioned within the tunnel of the PFO 5. As the elongated member 14 transitions from a first position to a second position the elongated member 14 deploys and branches into the two shafts 52a and 52b. The two shafts 52a and 52b expand the PFO tunnel laterally so the PFO tissues are apposed or are at least closer to each other. The elongated member 14 is then withdrawn proximally from within the tunnel of the PFO 5 while the abrasive materials 50 on the surface of the shafts 52a, 52b of the elongated member 14 abrade the tissues in the PFO tunnel. Energy is directed continuously or, alternatively, intermittently from the electrode 22 distal to the abraded tissues thereby inducing tissue adhesion from the distal end 102 of the PFO tunnel towards the proximal end 103 of the tunnel of the PFO 5.

After the elongated member 14 exits the PFO tunnel, it is withdrawn back into the lumen 24 of the catheter 12 to return the distal end portion 38 of the elongated member 14 to its first position housed within the catheter 12. The delivery system 8 is then withdrawn from the patients body.

In another embodiment of the method of the invention, the delivery system 8 includes an elongated member 14 including abrasives 50 such as the elongated members 14 with abrasives illustrated in FIGS. 8 and 9 and described in the corresponding text. As the elongated member is withdrawn, the tissues within the tunnel of the PFO 5 are abraded followed by the intermittent or continuous application of energy from one or more electrodes 22 as the elongated member 14 is withdrawn from the tunnel of the PFO 5.

The foregoing method may be altered in any number of ways without departing from the scope of the invention. For example, application of suction to appose tissues is not required in all embodiments. The exemplary method and embodiments of the system described herein are directed to closing a PFO but may be used for other tissue welding applications, e.g., closing an intraventricular or interatrial septal defect, other cardiac defects, or closure of the left atrial appendage. Furthermore, a variety of different energy types may be applied from a variety of different configured energy transmission devices. In some embodiments, one or more of the steps described above may be repeated one or more times. Moreover, any of the embodiments of the apparatus for closing a PFO described herein or any apparatus suitably configured to apply energy within the tunnel of or any defect characteristic of a PFO may be used according to the method described herein. Thus, the description of the method is provided for exemplary purposes only.

Variations, modifications, and other implementations of what is described herein will occur to those of ordinary skill in the art without departing from the spirit and the scope of the invention. The invention is not to be defined only by the preceding illustrative description.

What is claimed is:

1. A method for substantially sealing the tunnel of a PFO in the cardiac tissues of a patient, comprising:
introducing an apparatus into the heart comprising a catheter having an energy delivery element;
introducing the energy delivery element into the tunnel of the PFO;
applying energy from the energy delivery element to the tissues of the tunnel of the PFO while the energy delivery element is withdrawn from the tunnel.

2. The method of claim 1 wherein the energy from the energy delivery element is intermittently applied as an energized and de-energized cycle as the energy delivery element is withdrawn.

3. The method of claim 1 wherein the energy applied from the energy delivery element is continuously applied as the energy delivery element is withdrawn.

4. The method of claim 2 wherein the intermittent application of energy to the tissues of the tunnel comprises more than one energized and de-energized cycle.

5. The method of claim 1 wherein the energy delivering element delivers energy selected from the group consisting of radio frequency, cryogenic, laser, ultrasonic, resistive heat and microwave.

6. The method of claim 1 further comprising abrading the tissues in the tunnel of the PFO.

7. The method of claim 1 further comprising applying negative pressure from the catheter and stabilizing the end of the catheter on the tissues surrounding the opening of the PFO tunnel.

8. An apparatus for closing the tunnel of a PFO in a patient's heart, comprising:
a catheter comprising a lumen and extending from a proximal end to a distal end;
an elongated member comprising an electrode and extending from a proximal end to a distal end, and slideably movable, and axially disposed in the lumen of the catheter, said elongated member comprising a plurality of filaments, each filament comprising a fixed end and a free end, said fixed end joined to a distal end portion of said elongated member; and,
at least one electrode disposed on said filament.

9. The apparatus of claim 8 wherein said electrode is disposed on the free end of said filament.

10. The apparatus of claim 8 wherein said plurality of filaments are disposed on the distal tip of said elongated member.

11. The apparatus of claim 8 further comprising a vacuum cone disposed at the distal end of the catheter for applying a negative pressure to the patient's cardiac tissues surrounding the PFO.

12. The apparatus of claim 8 further comprising an electrode disposed at the distal tip of said elongated member.

13. The apparatus of claim 8 wherein said filaments are curvilinear.

14. The apparatus of claim 8 wherein said filaments are flexible.

15. The apparatus of claim 8 wherein said fixed end of each of said filaments is equidistant from the distal tip of said elongated member.

16. The apparatus of claim 8 wherein said distal end portion comprises 30% of the length elongated member at the distal end of the elongated member.

17. The apparatus of claim 8 wherein said distal end portion comprises 20% of the length elongated member at the distal end of the elongated member.

18. The apparatus of claim 8 wherein said distal end portion comprises 15% of the length elongated member at the distal end of the elongated member.

19. The apparatus of claim 8 wherein the fixed end of said filaments is joined to the distal tip of said elongated member.

20. The apparatus of claim 8 wherein the fixed ends of one filament is disposed at a first distance from the distal tip of the elongated member and the fixed end of another filament is disposed at a second distance from the distal tip of the elongated member.

21. The apparatus of claim 11 further comprising a vacuum source.

22. The apparatus of claim 8 further comprising an energy source.

23. The apparatus of claim 8 wherein said electrode delivers radio frequency energy.

24. A method for substantially sealing the tissue of a patent foramen ovale, comprising:
introducing an apparatus into the heart via the percutaneous, transvascular route, the apparatus comprising a catheter comprising a lumen and extending from a proximal end to a distal end;
a vacuum cone disposed at the distal end of the catheter for applying a negative pressure to the patient's cardiac tissues surrounding the PFO; and,
an elongated member comprising an electrode and extending from a proximal end to a distal end, and slideably movable, and axially disposed in the lumen of the catheter;
contacting the cone with and applying a negative force to the patient's intracardiac tissues;
introducing at least the distal tip of the elongated member into the tunnel of the PFO;
applying energy to the tissues within the tunnel;
removing the apparatus from the patient
